(19) United States
(12) Patent Application Publication
(10) Pub. No.: US 2007/0020316 A1
(43) Pub. Date: Jan. 25, 2007

(54) Personal topical disinfectant device

(76) Inventors: Raviv Laor, Philadelphia, PA (US); Caroline Laor, Philadelphia, PA (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 11/485,938

(22) Filed: Jul. 13, 2006

Related U.S. Application Data
(60) Provisional application No. 60/698,818, filed on Jul. 13, 2005.

(51) Int. Cl. A61K 9/70

(52) U.S. Cl. 424/443

(57) ABSTRACT

A personal topical disinfectant device, including a pair of the flexible, fluid-impermeable sheets joined at their peripheries to establish a fluid-tight central region in closing a folded flexible sheet impregnated with and ingestible antibacterial agent. Optionally, the flexible sheet is impregnated with a flavoring and/or texturizing agent.

### FIELD OF THE INVENTION

The field of the invention is disinfection devices, and more particularly, is directed to personal topical disinfectant devices, which eliminate bacteria and other microbes and reduce or eliminate body odor.

### BACKGROUND OF THE INVENTION

Packaged towelette-based personal topical devices are known in the art. See, for example, U.S. Pat. No. 3,972,360, which provides a towel impregnated with a self-tanning composition for application to a users scan. Present personal hygienic topical antibacterial agents are distributed in several forms, primarily as wipes (and towelettes), liquids, or aerosols for topical application. These products, though safe and effective as disinfectants, include ingredients which are not suitable for ingestion. In fact, many such devices contain warnings contra-indicating swallowing, and, in the case of ingestion, are to be treated as poisons.

### BRIEF DESCRIPTION OF THE INVENTION

The present invention is a device in the form of an injectable antibacterial composition, preferably with a flavoring, in or all in a substrate such as a towelette. The present invention combines the features of a disinfectant with the additional quality of being ingestible. The primary benefit of a device embodying the invention, is its antibacterial and deodorant features. In addition, in the event children or pets, pick up items that have been treated with the device, or kiss or lick the person who has applied the device to a portion of the person's body, then there would be no harm to the person or animal, even in the event some of the composition is ingested.

The temm “towelette” refers to a flexible sheet medium, which is packaged in “moist” form, within a pair of opposed fluid-impermeable sheets, for example, polyethylene-lined paper, or foil, with the sheets being joined at their peripheries, for example by crimping. A towelette is not new per se, neither is a towelette impregnated with antibacterial and deodorant compositions, however, a towelette impregnated with an ingestible antibacterial agent is not taught or suggested in the prior art.

A personal topical disinfectant device in accordance with the invention includes
A. a flexible and fluid-impermeable first sheet, said first sheet of having an associated two-dimensional shape and size,
B. a flexible and fluid-impermeable second sheet, said second sheet having an associated two-dimensional shape and size which substantially matches the shape and size of said first sheet, wherein said first and second sheets each include a peripheral region disposed about a central region thereof, and wherein said first sheet and said second sheet are disposed opposite each other with said peripheral regions being mutually joined, to define a fluid-tight capsule region, and
C. a flexible, sheet impregnated with an ingestible antibacterial agent, said impregnated flexible sheet being disposed in said capsule region.

In a preferred form, the antibacterial composition is flavored and includes certain glycinides to make it both tasty and have a pleasant texture on the tongue.

The compact towelette of the invention is convenient to have with a user anywhere he or she may be, for example, in the glove compartment of the users car, purse, or wallet, sports bag, office drawer, etc. Application is simple—the user opens the package, unfolds the antibacterial agent-laden flexible sheet and wipes it over the body portion, face, neck, etc., or item to be disinfected.

### DESCRIPTION OF THE DRAWING

FIG. 1 shows in perspective form, a personal topical disinfection device, according to an embodiment of the invention.

FIG. 2 shows in perspective form, the flexible sheet, impregnated with disinfection and flavoring, agents of the device of FIG. 1.

### DETAILED DESCRIPTION OF THE INVENTION

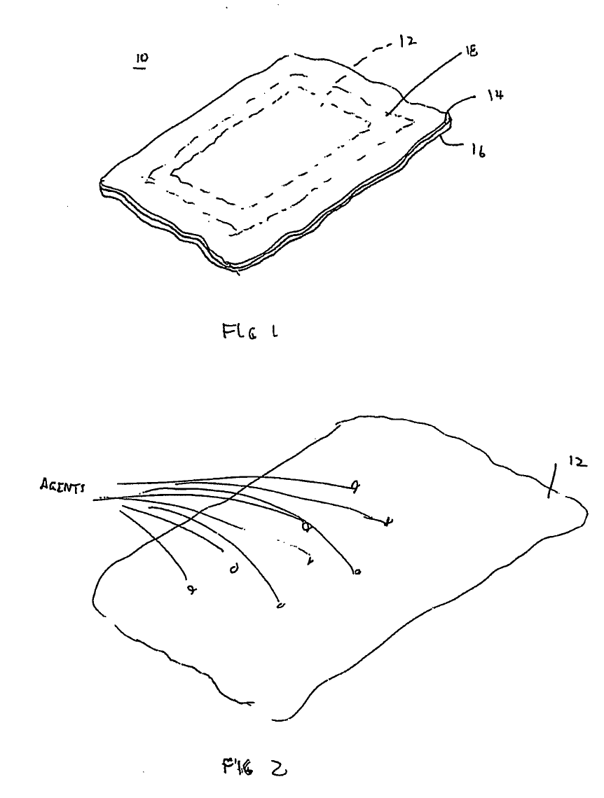

An exemplary personal topical disinfection device 10, embodying the invention is shown in FIG. 1 and FIG. 2. The device 10 is in the form of a packaged towel in which a flexible sheet 12 in a folded configuration, is disposed between a fluid-impermeable upper sheet 14 and a fluid-impermeable lower sheet 16, which sheets are joined at their peripheries to establish a fluid-tight chamber 18 enclosing the folded flexible sheet 12. By way of example, the sheets 14 and 16 may be paper (lined with polyethylene) or plastic or metal foil, and may be joined at their peripheries by a ceiling agent, or by crimping. The flexible sheet 12 may be made from paper or a woven textile, natural or synthetic, or a combination of natural and synthetic materials. In the illustrated embodiment, the sheets 14 and 16 are rectangular, but other shapes, polygonal or otherwise, may be used in other embodiments.

The folded sheet 12, prior to encapsulation by sheets 14 and 16, is impregnated with an antibacterial agent that is ingestible. Optionally, the flexible sheet may be impregnated with one or more flavoring agents, and in some cases, a texturizing agent. Conventional flavoring agents may be used, such a citrus flavoring. By way of example, an exemplary antibacterial agents, and an exemplary texturing agent are set forth in Table 1, but alternative agents for effecting these respective functions may be used in other embodiments.

Table 1
Disinfectant agent: Triclosan
Texturizing agent: Glycinide

The invention may be embodied in other specific forms without departing from the spirit or central characteristics thereof. The present embodiments are therefore to be considered in all respects as illustrative and not restrictive, the scope of the invention being indicated by the appended claims rather than by the foregoing description, and all changes that come within the meaning and range of equivalency of the claims, are therefore intended to be embraced therein.

1. A personal topical disinfectant device comprising:
A. a flexible and fluid-impermeable first sheet, said first sheet of having an associated two-dimensional shape and size,
B. a flexible and fluid-impermeable second sheet, said second sheet having an associated two-dimensional shape and size which substantially matches the shape and size of said first sheet, wherein said first and second sheets each include a peripheral region disposed about a central region thereof, and wherein said first sheet and said second sheet are disposed opposite each other with said peripheral regions being mutually joined, to define a fluid-tight capsule region, and
C. a flexible, sheet impregnated with an ingestible antibacterial agent, said impregnated flexible sheet being disposed in said capsule region.

2. A personal topical disinfectant device according to claim 1, wherein said first and second sheets are rectangular.

3. A personal topical disinfectant device according to claim 1 wherein said sheets are paper lined with plastic.

4. A personal topical disinfectant device according to claim 3, wherein the plastic is polyethylene.

5. A personal topical disinfectant device according to claim 1, wherein said flexible sheets are metal foil.

6. A personal topical disinfectant device according to claim 1, wherein said flexible sheets are plastic.

7. A personal topical disinfectant device according to claim 1, wherein said antibacterial agent is Triclosan.

8. A personal topical disinfectant device according to claim 1, wherein said flexible sheet is further impregnated with a flavoring agent.

9. A personal topical disinfectant device according to claim 8, wherein said flavoring agent is a citrus flavoring agent.

10. A personal topical disinfectant device, according to claim 1, is further impregnated with a texturizing agent.

11. A personal topical disinfectant device, according to claim 10, wherein said texturizing agent is a glycinide.
